(19) United States
(12) Patent Application Publication
(10) Pub. No.: US 2016/0199665 A1
(43) Pub. Date: Jul. 14, 2016

(54) TREATMENT OF WOUNDS USING ELECTROMAGNETIC RADIATION

(71) Applicant: PhotoMed Technologies, Inc., Kensington, CA (US)

(72) Inventors: Allan Gardiner, Kensington, CA (US); Jake Gardiner, Kensington, CA (US)

(21) Appl. No.: 14/592,505

(22) Filed: Jan. 8, 2015

(51) Int. Cl. A61N 5/06; A61B 5/00

(52) U.S. Cl. CPC A61N 5/0613; A61B 5/0077; A61N 2005/0659; A61N 2005/0661; A61N 2005/0663; A61N 2005/0636

(57) ABSTRACT

This invention provides methods for treating wounds using localized electromagnetic radiation directed at excitable tissues, including affected tissues, nerves, muscles, and blood vessels. By controlling the wavelength, the wavelength bandwidth, pulse duration, intensity, pulse frequency, and/or variations of those characteristics over time, and by selecting sites of exposure to electromagnetic radiation, improvements in the function of the different tissues and organs can be provided. Responses can be monitored by using visible and nonvisible characteristics of wounds. Changes in wound characteristics can become more visible under RGB and blue light wavelengths.

### FIELD OF THE INVENTION

This invention relates to methods for treating wounds using electromagnetic radiation. More particularly, this invention relates to applying electromagnetic radiation having controlled wavelengths, bandwidths, pulse durations, pulse frequencies and/or intensities applied to areas of the body associated with a wound.

### BACKGROUND

Chronic wounds are a major public health concern, affecting more than 6 million people in the United States alone. The number of chronic wounds are rising along with diabetes, obesity, and aging. Some co-morbidities, such as pain and peripheral artery disease, can increase wound healing times. Wounds can result from inactivity, such as being confined to bed in a hospital or convalescent home. The standard of care is often inadequate, with many chronic wounds lasting for years.

### SUMMARY

We have identified a new approach to treating wounds. Laser and LED methods are limited by using discrete wavelengths, which are inherent in these technologies.

In contrast, methods of this invention improve on the effectiveness of light-based therapy by using non-coherent electromagnetic radiation and expanding the number of wavelengths applied to the wound. This increases the number of wavelength-dependent wound factors stimulated. Also, by varying the wavelength during therapy, the wound is constantly presented with a changing stimulus that prevents habituation,

Changes in wounds become more visible under certain wavelengths. Fresh blood is typically red for example, so viewing it under yellow, green, or blue light can make it more visible than under red or full-spectrum light. Other wound factors also become more apparent under certain wavelengths. Changing the wavelength during therapy allows the clinician to better observe changes to the wound in real time. Also, separating photos or videos into red, green, and blue channels can highlight changes in different wound factors. These detection methods can also work with non-varying wavelength treatments, such as from lasers or LEDs.

Thus, one object of this invention is the development of improved methods for treating disorders of the body using electromagnetic radiation, and in particular, wounds and/or pain associated with wounds.

### BRIEF DESCRIPTION OF THE FIGURES

This invention is described with reference to specific embodiments thereof. Other features can be appreciated with respect to the figures, in which:

FIGS. 1A through 1I depict photographic images of a wound exposed to broad spectrum non-coherent radiation viewed using different channels. FIGS. 1A, 1B, 1C depict the wound viewed with standard RGB channels. FIGS. 1A, 1D, and 1G depict images taken before treatment. FIGS. 1B, 1E, and 1H were taken at the end of a treatment 6 min later. FIGS. 1C, 1F and 1I depict images taken after an additional 12 minutes. FIGS. 1D, 1E, and 1F, show that changes in the wound were less visible when viewed using the red channel only. FIGS. 1D, 1E, and 1F depict images viewed with the blue channel alone.

### DETAILED DESCRIPTION

These and other objects are met by methods of this invention for therapeutic application of electromagnetic radiation to tissues that are sensitive to such radiation. Therapeutic aims include normalization of blood flow to and from, and lymphatic flow from affected regions, and normalization of muscle tone, nerve activity and other tissue functions. Specific wavelengths can be chosen based on physiologic screening and sensitivity testing conducted prior to and during the application of treatment. Monitoring of the patient's condition can be selected based on the patient's specific diagnosis and the organ systems and tissues affected.

Electromagnetic radiation therapy can be carried out by exposing a site on the body with localized non-coherent radiation of a desired peak wavelength and wavelength bandwidth (herein known as “bandwidth”) which does not vary over time, including those in the infrared, visible, ultraviolet and other portions of the electromagnetic spectrum. Additionally, the wavelength used can vary over time (VOT). Fiber optics or other types of waveguides can direct beams of electromagnetic radiation to specific, pre-defined sites on a body with ease. Additionally, with the advent of devices incorporating dual or multiple illumination systems U.S. Utility Pat. Nos. 6,886,984, 7,180,802, 7,720,306, 7,878,965, 7,918,779, 8,343,026, each patent incorporated herein fully by reference), it is now possible to provide independently controlled beams of electromagnetic radiation to specific locations. In other aspects of this invention, beams of electromagnetic radiation can be used either simultaneously or sequentially, each having separately controllable wavelength, bandwidth, intensity, pulse duration, pulse frequency, phase, or polarization.

For example, the central wavelength of a narrow bandwidth beam can vary from about 300 nm to about 1100 nm. The difference between the minimum and maximum wavelengths can be from about 1 nm to the full range (800 nm). The bandwidth can vary from about 1 nm to about 200 nm, The time to change any controllable variable from minimum to maximum, or vice versa, can be from about 1 second to the full duration of the application. The term “about” herein refers to a range around the value of the variable±20% of the value.

It can also be appreciated that one can have variations that are asymmetrical. For example, the wavelength can change faster in one direction than the other. Additionally, the rate of change can be varied to provide linear, sinusoidal, trapezoidal, or other output.

One or more methods for selecting and/or varying wavelength and/or wavelength variation over time can be used. For example, prisms, diffraction gratings, rulings, or filters can used. Tunable lasers, tunable LEDs, diode array emitters, or other technologies can also be used.

In addition applying electromagnetic radiation directly to skin or oral wounds, other areas of a subject's body can be illuminated. For example, trigger points, acupuncture points, electro-diagnostic points, nerve distributions, or blood vessels can be illuminated alone or in combination. Additionally, to improve transparency of the subject's skin, a small drop of liquid can be used, such as water or oil.

Improved methods for evaluating effects of electromagnetic radiation therapy on wounds include, but are not limited to, observation of changes in wound factors at different wavelengths, different color channels of imaging devices, and Doppler blood flow. Methods for evaluating effects on pain include, but are not limited to, the use of sensitive infrared cameras to monitor changes in body surface temperature (“thermography”), surface electromyography (“sEMG” or “SEMG”), oximetry, pulse volume, tissue compliance, monofilament testing, Doppler blood flow, pressure threshold, current perception threshold, electro-dermal activity (“EDA”; a measurement of skin conductance), sweat tests such as the Alizarin Sweat Test, somatosensory testing, heart rate variability (including entrainment), nerve conduction velocity, campimetry, algorimetry, and other methods described herein below and those known in the diagnostic and/or evaluative arts.

To treat peripheral symptoms with electromagnetic therapy, it can be desirable to expose a nerve innervating that site close to the exit of the nerve from the central nervous system (a “proximal” location). It can be desirable to expose a more peripheral part of the nerve (a “distal” location). Alternatively, it can be desirable to expose a nerve in an intermediate position between a distal site and a proximal site. Further, it can be desirable to simultaneously expose different locations of the same nerve to electromagnetic radiation, and in further embodiments, it can be desirable to expose nerves to radiation at different times in different locations.

To treat central nervous system disorders, it can be desirable to modify the activity of sensory afferent nerves. Alterations in sensory nerve activity can occur within structures in the spinal cord and/or the brain, including those structures that are responsible for pain transmission, motor function, or motor control.

### Methods for Accelerating Wound Healing

Methods of this invention can be used to treat many different kinds of wounds. Wounds include open wounds, ulcers, infections, bruises, inflammation, pain, phantom pain, itching, eczema, cellulitis, other skin disorders, dental disorders, stem cell activation, and other wounds.

We were initially surprised when fresh blood appeared in a wound area partway through a two-minute treatment. The blood was visible when the wavelengths were in the yellow portion of the spectrum (about 580-610 nm) but was not visible when illuminated with red light. Later, other changes to the wound were also documented, such as flattening, wrinkling, wound texture, granulation, and scabbing. We conclude that wound healing can be accelerated using methods of this invention.

One feature of certain aspects of this invention is the variation of wavelength over time. Once a wavelength range and time of application of light are chosen, the rate of change of wavelength can be controlled. For example, the wavelength can change from about 1 nm/sec to about 100 nm/sec.

Additionally, the power output of an illuminator can be varied. In some embodiments, the power can be in about 1 milliWatt (mW)/cm². In other embodiments, the power can be from 1 mW to about 200 mW/cm². In other embodiments, the power output is limited only by comfort, heating of the tissues treated, and the size of the treated area.

In other aspects, the bandwidth can be adjusted to provide illumination around a central wavelength of, for example, from 10 nm to about 100 nm. In some embodiments, the bandwidth can be limited to about 10 nm around a central wavelength to provide a range of wavelengths broader than a laser-based system.

The efficacy of therapy using methods can depend on the type of injury, the magnitude of tissue damage, the depth of the injury, pre-existing conditions of the patient, and other factors. In some cases, it can be desirable to expose a wound to electromagnetic radiation having central wavelengths in the range of about 300 nm to about 1100 nm. For treatment of fresh wounds associated with bleeding or weeping of extracellular fluid, wavelengths in the blue-green (400 nm to about 550 nm) can be useful. In other circumstances, treatment of the periphery of a wound can be exposed to light in the yellow to red (about 560 nm to about 700 nm). In other cases, wounds can be exposed to wavelengths from about 400 nm to about 700 nm.

Efficacy of treatment can be evaluated using observation of characteristics of the wound. For example, the wound can weep colored fluid (exudate) that is visible at some wavelengths yet not at others. For example, blood can be visible in the blue, green, and yellow wavelengths, but not in the red or infrared portion of the spectrum. Additionally, changes in texture and/or elevation of the wound can be observed. Later, one can observe changes in the margins or scab formation of the wound and granulation can become apparent.

A typical patient may present with a slow or non-healing wound that may have persisted for months or years in the face of traditional wound care. Other patients may have fresh injuries caused by surgery or accidents.

Some patients experience pain associated with a wound. Examples include itching and phantom pain. Therapy using methods disclosed herein can alleviate the pain as well as aid in healing.

Wavelengths in the ranges of blue (about 430 nm to about 490 nm), violet (about 400 nm to about 430 nm), or ultraviolet (about 300 nm to about 400 nm) can be used to inhibit bacterial growth, thereby combatting infections that can be present with many types of wounds.

### EXAMPLES

The examples that follow represent specific studies that we performed on patients with wounds. It can be appreciated that applications of the methods described herein can be applied to other patients, and to other types of wounds. All such applications and embodiments are considered part of this invention.

### Example 1

### Treatment of a Wound Flap

A 58-year-old female (weight 84 kg, height 170 cm, Caucasian) in an IRB-reviewed study presented with a two-day-old wound on her right hand caused by a gouge from zipper pull-tab. She reported a history of fragile skin and slow wound healing. She estimated that a wound of this type would normally take her one to two months to heal with help from the wound care center.

A practitioner had cleaned and repositioned a 6 mm square skin flap that was connected to intact skin on only one side. Before treatment according to methods of this invention, much of the flap appeared dark gray. One side of the flap was bordered by a dry recessed gap, approximately 1 mm wide×0.5 mm deep.

During the first treatment using non-coherent electromagnetic radiation (590-690 nm, 5 nm/sec variation in wavelength, frequency of 0 Hz, for a duration of 120 seconds, spot size about 5 cm in diameter with a power setting of from about 5 mW/cm² to about 30 mW/cm²) applied directly to the wound, fresh blood filled the previously dry gap. The blood was clearly visible in the 590 nm range and difficult to observe in the 630 nm to 690 nm range. Within a few minutes, the flap color changed from gray to pink and the fresh blood formed a scab.

One week later, the wound flap had healed. The only remaining sign of the wound was a small, slightly depressed scab. The scab was completely gone a week later.

Over the next several months, she returned with other wounds and bruises. They also healed faster than she expected.

Example 2

### Slow-Healing Wound

A 48-year-old female (weight 102 kg, height 165 cm, Caucasian) in an IRB-reviewed study presented with a slow healing wound on her left buttock where a spinal cord stimulator had been surgically implanted five weeks before. Portions of the 4.5 cm long wound had reopened over infected internal stitches. She had undergone three courses of antibiotics.

She presented with the latest wound about two weeks old that had slowly healed to about an 8 mm diameter. A depression in the center was filled with fluid. She received a single treatment of electromagnetic radiation (wavelengths 590 nm to 630 nm, rate of change of wavelength 2 nm/sec, frequency 0 Hz, duration 120 seconds, spot size of about 2.5 cm (depending on the distance between the tip of the illuminator and the wound) and with a power density from about 5 mW/cm² to about 30 mW/cm² depending on the wavelength and the position of the light to the wound) directly to the wound. The subject and her husband reported that the color and character of the wound rapidly improved after treatment.

She returned 10 days later and received 7½ minutes of electromagnetic radiation as above, but with a wider wavelength range, with wavelengths varying between 430 nm and 690 nm. The wound changed color during therapy and a spot of fresh blood appeared in the center of the wound. She reported feeling itching at the site.

She returned four days later and reported that the wound was healing well and that her husband continued to be surprised by the rate of healing. No additional breakdowns in the wound had taken place. Three minutes of treatment was administered with wavelengths varying between 430 nm and 690 nm.

Two weeks later, the wound had healed and no additional therapy was needed. One year later, she reported that the wound had not reappeared.

### Example 3

### Foot Wound With Pain

A 68-year-old female (weight 116 kg, height 173 cm, Caucasian) in an IRB-reviewed study presented with an open wound about 1.5 cm×2 cm in diameter in the center of the plantar surface of her right foot. She also had swelling, redness, and pain of her right lower leg.

The subject's foot wound made walking painful, and accommodating the foot led to more pain in other locations. She received two exposures of the wound site to electromagnetic radiation, duration 2½ minutes, wavelengths varying between 580 nm and 690 nm, and for a duration of 1½ minutes at wavelengths varying between 490 nm and 690 nm, a spot size of about 2.5 cm, and a power density of about 5 mW/cm² to about 30 mW/cm². We observed dark spots forming in the wound area during treatment when the wavelengths applied were in the yellow wavelength range of about 580 nm to 610 nm. The wound appeared to dry and flatten after the treatment. She reported good pain relief by the end of the session.

One week later, the wound was noticeably smaller. In a second session, she was given electromagnetic radiation for a duration of 1½ minutes at wavelengths varying from 580 nm to 690 nm, and another exposure for a duration of 1½ minutes using wavelengths varying from 490 nm to 690 nm. She reported that her foot pain was further reduced as a result of the exposure.

The following week, more positive changes were seen in the wound. Treatments from the second session were repeated.

Three weeks after the original session, we made video recordings using two color cameras and a black and white camera during each treatment. Videos taken using each camera at the wavelengths 430 nm, 560 nm, and 690 nm were compared. We noticed changes in the wound during treatment. The changes were clearly distinguishable at 430 nm and 560 nm but not at 690 nm. High resolution, still images from the session also confirmed rapid healing. Similarly, the changes were clear in the green and blue channels of the Red Green Blue (RGB) image, but not in the red RGB channels.

One month later, the wound had flattened, healed and was no longer painful. The patient was very pleased with the relatively rapid healing in response to non-coherent electromagnetic radiation compared to her past, non-healing, and infected wounds.

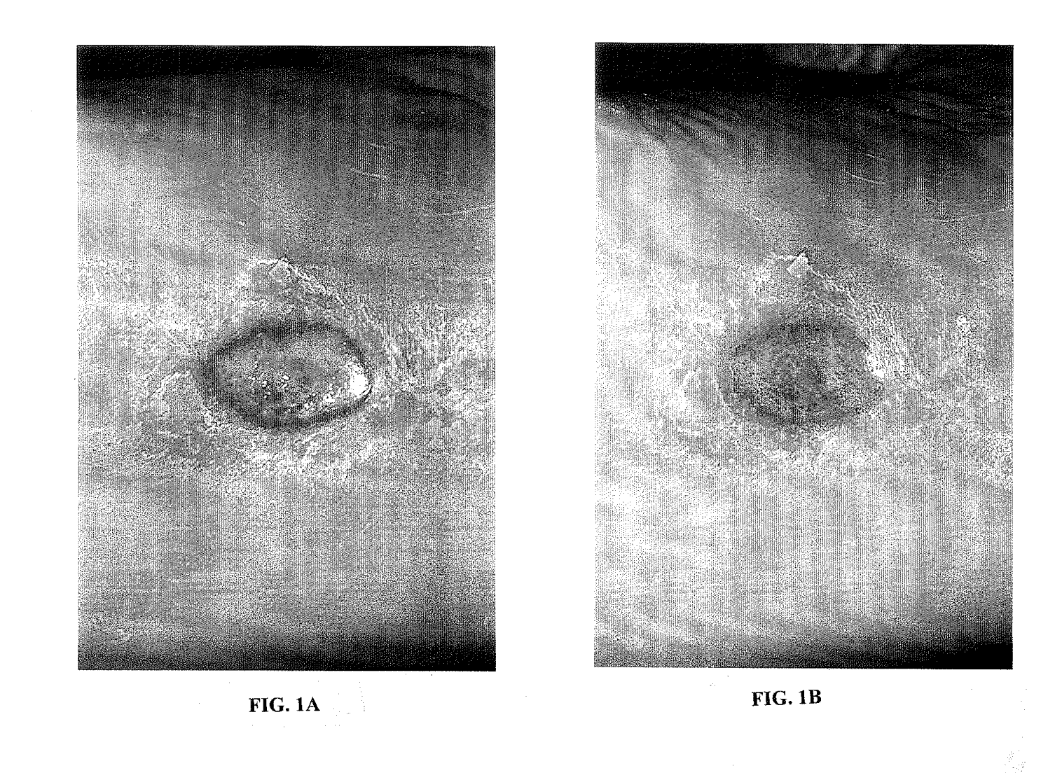

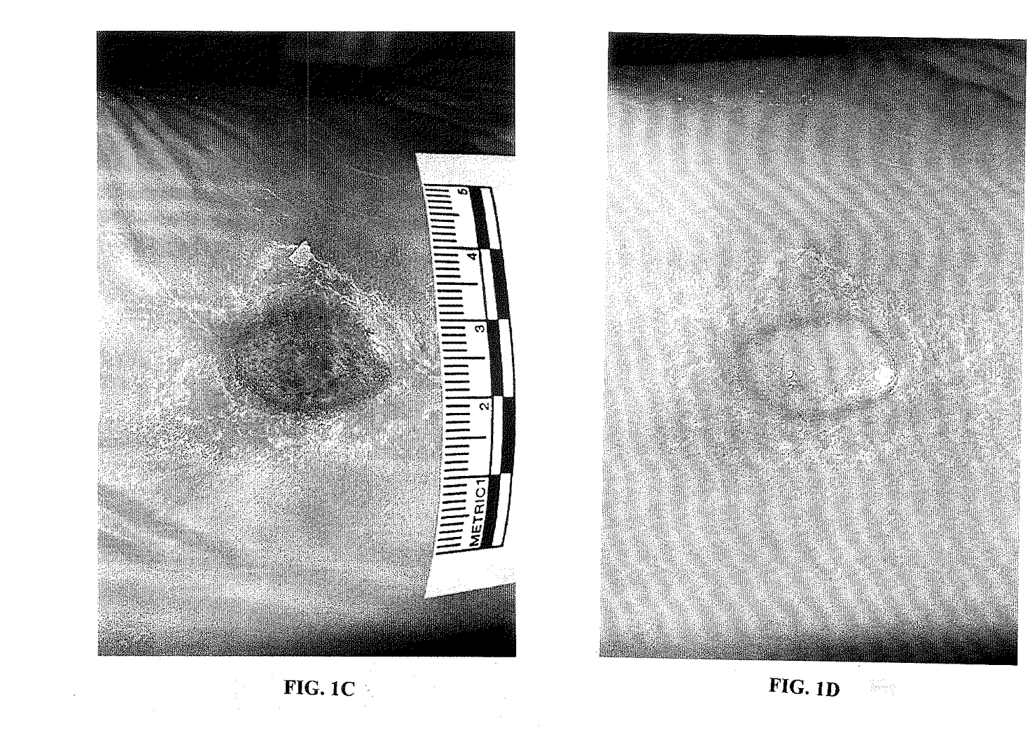

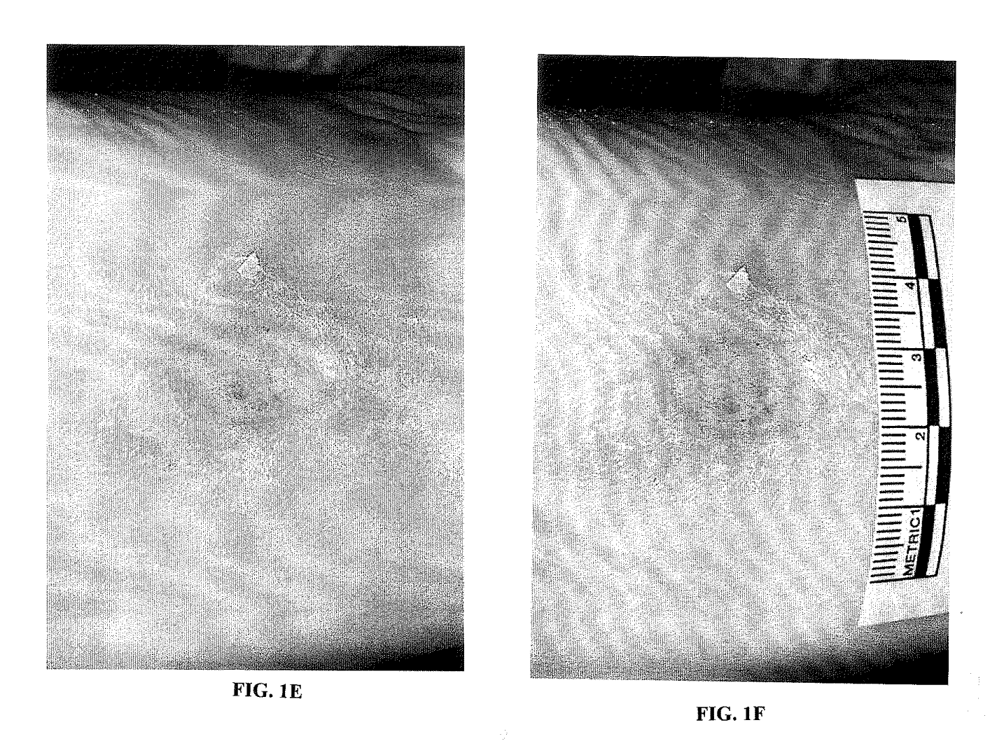

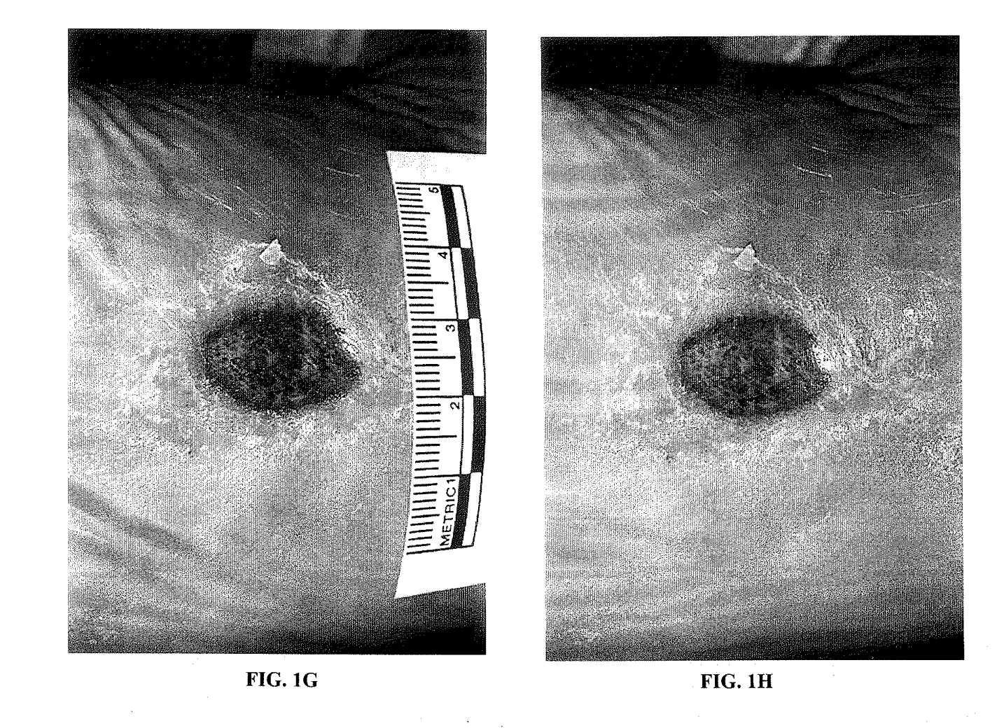

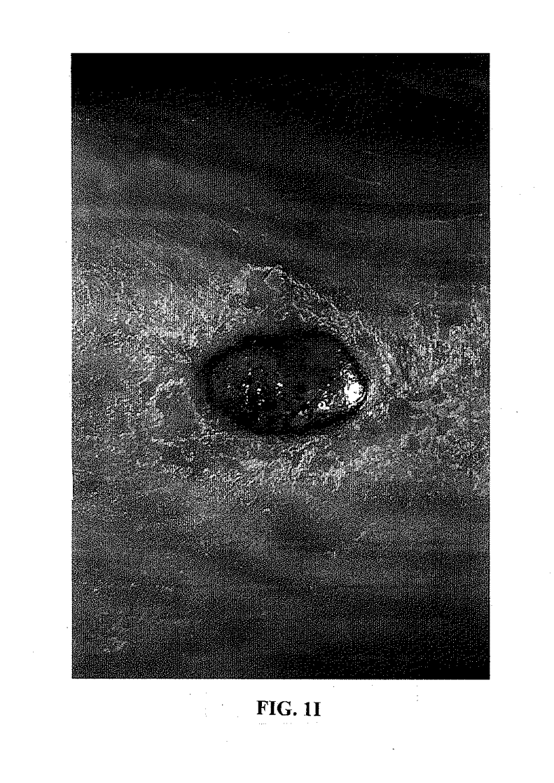

As can be seen in FIGS. 1A-1I, the plantar wound appears visible with standard RGB channel (FIGS. 1A, 1B, 1C). Images shown in FIGS. 1A, 1D, and 1G were taken before treatment. Images shown in FIGS. 1B, 1E, and 1H were taken at the end of a treatment 6 min later. Images shown in FIGS. 1C, 1F and 1I were taken after an additional 12 minutes. Unlike the appearance with standard RGB channel, when the wound was viewed with the red channel only (FIGS. 1D, 1E, and 1F), the wound was much less visible. In contrast with the images taken using the red channel alone (FIGS. 1D, 1E, and 1F), when viewed with the blue channel alone, (FIGS. 1G, 1H, and 1I), the wound was clearly visible.

### Example 4

### Treatment of Cellulitis

A 57-year-old female (weight 150 kg, height 165 cm, Caucasian) in an IRB-reviewed study presented with cellulitis of the right shin. She also had a history of diabetic neuropathy with her feet partially numb and burning in the afternoon. She had other problems related to weight gain after a reversal of a bariatric surgery. Her entire body was enlarged by edema that her many doctors were unable to relieve. Because her skin would frequently break down producing an open wound characteristic of cellulitis, she was continuously taking antibiotics to avoid deeper infection. Each wound required skilled wound-care nursing to avoid enlarging the wound. She reported that her wounds frequently took 2 to 3 weeks to seal and longer to heal.

She presented with a fresh 3 mm wide by 5 mm long wedge-shaped area of cellulitis. She received electromagnetic radiation for a duration of 80 seconds, wavelengths varying between 580 nm to 690 nm directly to the wound, and another treatment for a duration of 80 seconds using wavelengths varying from 430 nm to 550 nm, spot size of about 2.5 cm, power density of about 5 mW/cm² to about 30 mW/cm² directly to the wound. During treatment, a 2 mm diameter bead of clear wet fluid without redness appeared. We believe that the lack of redness indicated that no blood was present and the exudate was extracellular fluid. The bead of fluid remained clear at all wavelengths. There was a slight darkening in the margins of the wound as observed using yellow wavelengths. Her husband said the cellulitis did not look as shiny or taut at the end of the session. She received additional therapy for pain in her feet and right knee after the cellulitis treatment.

On her second visit, two weeks later, she reported that the area of cellulitis had scabbed and cleared in three days. There was no evidence of the wound beyond a slight darkening of the skin. She and her husband were surprised there had been no further episodes of cellulitis in the two weeks between sessions of therapy. She reported some relief in the cellulitis “stretched” feeling as well. Her wound care nurse was surprised the healing was so fast and had provided her with two weeks of drainage supplies that turned out not to be needed.

Two months later she was pleased to report that she had not had any more episodes of weeping cellulitis.

### Example 5

### Treatment of Eroded Gums

A 58-year-old female (weight 81 kg, height 165 cm, Hispanic) in an IRB-reviewed study presented with pain in her eroded gums caused by a pain medication that had induced loss of saliva. We applied electromagnetic radiation for a duration of 2½ minutes and with wavelengths varying between 580 nm and 690 nm, spot size of about 2.5 cm, with a power density of about 5 mW/cm² to about 30 mW/cm², delivered to the outside of her cheek. Subsequently she received another treatments for a duration of 1½ minutes with wavelengths varying from 430 nm to 550 nm, in four sessions over three months. She reported good pain relief and her gums healed sufficiently to no longer need treatment.

The following month she presented with a blister in her right palm. We applied electromagnetic radiation for a duration of 2½ minutes with wavelengths varying from 580 nm to 690 nm, and a subsequent exposure for a duration of 2 minutes at wavelengths varying from 430 nm to 580 nm. At the end of the session, she reported the blister felt drier, the itching had stopped, and she could open her hand fully without discomfort. At her next session, three weeks later, the blister was gone. She reported that it had healed much faster than she expected.

### Example 6

### Treatment of Eczema

A 76-year-old male (weight 75 kg, height 172 cm, Caucasian) in an IRB-reviewed study presented with eczema on his left knee that had been there for 4 months. He received one exposure to electromagnetic radiation directly to the affected area for a duration of 2 minutes with wavelengths varying from 580 nm to 690 nm, with a spot size of about 2.5 cm and a power density of about 5 mW/cm² to about 30 mW/cm² directly to the site of eczema. At a subsequent visit 10 days later, the eczema was gone. 10 months later, he reported that the eczema had not returned.

### Example 7

### Treatment of Orthodontic Dental Adjustment with Pain

A 34-year-old male (weight 77 kg, height 183 cm, Caucasian) had a temporomandibular joint (TMJ) disorder with moderate pain. To treat the pain, he had an orthopedic Advanced Lightwire Functionals (ALF) appliance and dental braces installed. Two days after installation of the ALF and braces, we applied electromagnetic radiation for approximately 10 minutes with the wavelength varying between 580 nm and 690 nm, a spot size of about 2.5 cm, and a power density of about 5 mW/cm² to about 30 mW/cm² to his mouth, head, and neck.

He reported feeling the muscles of his jaw, head and neck relax during therapy and less pain the following days. Over the next two weeks, we applied the same treatment 3 more times. His dentist and dental assistant both commented that the teeth had moved much more quickly than they expected. They expedited the tightening schedule. At the current time, he has less pain.

### Example 8

### Treatment of Phantom Pain Associated with Amputation

A 36-year-old female (weight 52 kg, height, 155 cm, Caucasian) in an IRB-reviewed study presented with complex regional pain syndrome (CRPS) and a more recent amputation of her left leg that was causing phantom pain. The remaining portion of her leg was sutured and one of the stitches had come loose, resulting in a current wound with severe pain at the wound and elsewhere, as typical of patients with CRPS.

We treated the wound for two minutes using light with wavelengths varying in the range of about 580 nm to about 690 nm, with a spot size of about 2.5 cm and a power density of about 5 mW/cm² to about 30 mW/cm².

During treatment, we noticed appearance of a glistening at the wound site, indicating exudation of extracellular fluid. Immediately after treatment, her pain at the wound site, phantom pain, and more generally associated with CRPS was reduced and she appeared more relaxed.

### CONCLUSION

Methods of this invention accelerate healing of open wounds, ulcers, infections, bruises, inflammation, pain, phantom pain, itching, eczema, cellulitis, other skin disorders, dental disorders, stem cell activation, and other wounds. Viewing wounds under different wavelengths or RGB channels can make changes more apparent.

1. A method for treating a wound in a mammal comprising of the steps:
exposing a tissue associated with a skin or oral wound to localized electromagnetic radiation having a central wavelength in the wavelength range of about 300 nm to about 1100 nm, a bandwidth within the wavelength range, and a controlled wavelength continuously varying over time;
said treating resulting in improved healing of the wound.

2. The method of claim 1, where said wound is of a hand.

3. The method of claim 1, where said wound is of a foot.

4. The method of claim 1, where said wound is of a surgical site.

5. The method of claim 1, said wound being associated with cellulitis.

6. The method of claim 1, where said wound is monitored or recorded using black and white, color, or infrared photography or videography to observe characteristics at different wavelengths.

7. The method of claim 1, further comprising use of electromagnetic radiation having a controlled variable selected from the group consisting of:
(i) pulse duration;
(ii) pulse frequency variation over time;
(iii) bandwidth;
(iv) intensity; and
(v) intensity variation over time.

8. The method of claim 1, where the central wavelength is within the blue-green wavelength range of about 400 nm to about 570 nm.

9. The method of claim 1, where the central wavelength is within the yellow-red wavelength range of about 570 nm to about 700 nm.

10. The method of claim 1, said treatment is in the wavelength range of about 300 nm to about 1100 nm, and said wound is observed using visible wavelengths.

11. The method of claim 10, said observing is carried out by separating an image of said wound into separate color channels.

12. The method of claim 11, said channels being Red, Green, and Blue (RGB).

13. The method of claim 12, said channel being Green wavelength about 490 nm to about 550 nm.

14. The method of claim 12, said channel being Blue having wavelengths in the range of about 400 nm to about 490 nm.

15. The method of claim 1, where said wound is monitored or recorded by a Doppler flow measuring device or imager.

16. The method of claim 1, where the localized treatment includes the entire body.

17. The method of claim 17, said continuous variation of wavelength over time being between about 1 nm/sec to about 100 nm/sec.

18. The method of claim 10, said wound is observed using visible wavelengths of about 400 nm to about 700 nm.
